(19) United States
(12) Patent Application Publication
(10) Pub. No.: US 2025/0041248 A1
(43) Pub. Date: Feb. 6, 2025

(54) ALZ-801 FOR USE IN TREATING ALZHEIMER'S DISEASE

(71) Applicant: Alzheon, Inc., Framingham, MA (US)

(72) Inventors: Susan Abushakra, Framingham, MA (US); Neil William Flanzraich, Coral Gables, FL (US); John Hey, Framingham, MA (US); Martin Tolar, Framingham, MA (US)

(21) Appl. No.: 18/717,139

(22) PCT Filed: Dec. 9, 2022
(86) PCT No.: PCT/US2022/052331

§ 371 Date: Jun. 6, 2024

Related U.S. Application Data
(60) Provisional application No. 63/287,552, filed on Dec. 9, 2021.

(51) Int. Cl. A61K 31/16; A61P 25/28

(52) U.S. Cl. CPC A61K 31/16; A61P 25/28

(57) ABSTRACT

Provided herein are methods for treating Alzheimer's disease using increased dosages amounts of ALZ-801.

### RELATED APPLICATIONS

This application claims the benefit of priority to U.S. Provisional Application No. 63/287,552, field Dec. 9, 2021, the entire contents of which are incorporated herein by reference.

### BACKGROUND

Alzheimer's disease (AD) is currently ranked as the sixth leading cause of death in the United States. The symptoms of AD worsen over time, although the rate at which the disease progresses can vary. The stages of AD are separated into three categories: early, middle, and late AD (also commonly referred to as mild, moderate, and severe AD). Since Alzheimer's affects people in different ways, each person may experience symptoms or progress through the stages differently.

ALZ-801, a promising new treatment for AD, is currently being investigated in clinical trials for subjects with early AD (MMSE>22). Initial data using an oral dose of 265 mg ALZ-801 twice daily (BID) showed about a 125% benefit in cognition (ADAS-cog) and an 81% benefit in function (CDR-SB) compared to placebo. See e.g., Tolar, et al., Int. J. Mol. Sci. 2021, 22, 6355. ALZ-801 has good blood brain barrier penetration and the ability to target toxic soluble amyloid oligomers.

Despite these benefits, there remains a need for treating patients who have moderate to severe AD (MMSE<22).

### SUMMARY

It has now been found that dosage amounts of ALZ-801 exceeding 530 mg/day (e.g. 265 mg BID) may elicit disease modifying effects in subjects with moderate to more severe AD. Evidence of this newly found effect has been observed in a recent compassionate use study being conducted under an Expanded Access IND. See e.g., the compassionate use study below where a 73 year old female subject diagnosed with major neurocognitive disorder of the Alzheimer type (i.e., MMSE believed to be <22 based upon symptoms) initially showed symptom improvement and stabilization following treatment with 265 mg ALZ-801 BID, but then showed a regression in cognitive improvement and/or related symptoms following, or at the tail end of, a 14 month treatment period. Remarkably, upon increasing the subject's dose of ALZ-801 from 265 mg BID to 265 mg TID, there was a rapid and clear improvement in the subject's motivation, verbal output, physical activity, activities of daily living (i.e. cooperative interactions with caretakers, ability to perform minor tasks, etc), mood, and gait. The higher dose was also well-tolerated. This initial period of clinical improvement lasted for approximately a year after which time the subject started showing a decline characterized by worsening cognition, apathy and general decreased activity. After a total of about fifteen months on ALZ-801 265 mg TID, the subject dosage was increased to 265 mg QID (four times daily). The subject has been receiving this QID dosing for the past six months. As with the TID dosing, the QID dosing was well tolerated and the subject improved over the later stages of the TID dose regimen with increased alertness, greater responsiveness and a more positive disposition.

Therefore, in one aspect, provided are methods for treating Alzheimer's disease in a subject having moderate to more severe AD (e.g., an MMSE score below 22) by administering to the subject at least about 700 mg/day of ALZ-801.

In other aspects, provided are methods for treating Alzheimer's disease by a) administering a first dosage of ALZ-801 of about 400 mg/day to about 600 mg/day (e.g., about 530 mg/day) to a subject for a period of about 50 weeks or more, or until the subject demonstrates a decreased therapeutic response to the first dosage; and b) administering a second dosage of ALZ-801 that is at least 1.5 times greater than the first dosage (e.g., at least about 700 mg/day) thereafter.

In certain aspects, the subject is administered a third dosage that is at least 2 times greater than the first dosage (e.g., at least about 1000 mg/day) when the subject demonstrates a decreased therapeutic response to the second dosage.

Further provided are methods for treating Alzheimer's disease is a subject using at least about 700 mg/day ALZ-801 such as about 265 mg TID or at least about 1000 mg/day ALZ-801 such as about 265 mg QID.

In certain aspects, the subject to be treated by the present methods has an MMSE score below a certain threshold or between a particular range.

In certain aspects, the subject to be treated by the present methods has moderate to severe AD. In other aspects, the subject to be treated by the present methods had mild AD and has progressed to a worsened degree of mild AD, or to moderate or severe AD (e.g., as determined by one or more cognitive tests or other methods). In other aspects, the subject to be treated by the present methods had moderate AD and has progressed to severe AD.

In certain aspects, the subject to be treated by the present methods is APOE4 positive.

### BRIEF DESCRIPTION OF THE DRAWINGS

FIG. 1A is a graph showing the steady state tramiprosate plasma AUC for different frequencies of ALZ-801 dosing.

FIG. 1B is a graph showing the and Cₘₐₓ for different frequencies of ALZ-801 dosing.

### DETAILED DESCRIPTION

As part of a first embodiment, provided herein is a method of treating Alzheimer's disease in a subject comprising the step of administering to the subject at least about 700 mg/day (e.g., about 795 mg/day) ALZ-801.

ALZ-801 refers to valyl-3-amino-1-propanesulfonic acid, represented by the structure below:

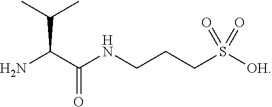

The term “subject” and “patient” are used interchangeably. In one aspect, the subject is a human. In some aspects, the subject is a human aged 100 years old or less, 95 years old or less, 90 years old or less, or 85 years old or less. In other aspects, the subject is a human aged 65-100 years old, 65-95 years old, 65-90 years old, or 65-85 years old. In yet other aspects, the subject is human age 58 years old or older. In still other aspects the human is in need of treatment.

As used herein, the term “treat”, “treating” or “treatment” means reversing, alleviating, inhibiting, or slowing the progress of Alzheimer's disease (AD), including cognitive decline or one or more symptoms associated therewith.

As part of a second embodiment, provided herein is a method of treating Alzheimer's disease in a subject comprising the steps of: a) administering about 400 mg/day to about 600 mg/day (e.g., about 530 mg/day) of ALZ-801 to the subject for a period of about 50 weeks or more; and b) administering at least about 700 mg/day (e.g., about 795 mg/day) of ALZ-801 thereafter. Alternatively, as part a) of the second embodiment, the subject is administered about 400 mg/day to about 600 mg/day (e.g., about 530 mg/day) of ALZ-801 to the subject for a period of about 52 weeks or more. In another alternative, as part a) of the second embodiment, the subject is administered about 400 mg/day to about 600 mg/day (e.g., about 530 mg/day) of ALZ-801 to the subject for a period of about 55 weeks or more, about 60 weeks or more, about 65 weeks or more, about 70 weeks or more, about 75 weeks or more, or about 80 weeks or more. In another alternative, as part a) of the second embodiment, the subject is administered about 400 mg/day to about 600 mg/day (e.g., about 530 mg/day) of ALZ-801 to the subject for a period of about 50 weeks to about 80 weeks, about 50 weeks to about 75 weeks, about 50 weeks to about 70 weeks, about 50 weeks to about 65 weeks, about 52 weeks to about 80 weeks, about 52 weeks to about 75 weeks, about 52 weeks to about 70 weeks, or about 52 weeks to about 65 weeks.

As part of a third embodiment, provided herein is a method of treating Alzheimer's disease in a subject comprising the steps of: a) administering about 400 mg/day to about 600 mg/day (e.g., about 530 mg/day) ALZ-801 to the subject until the subject demonstrates a decreased therapeutic response to such administration; and b) administering at least about 700 mg/day (e.g., about 795 mg/day) of ALZ-801 thereafter.

In some aspects, of the first, second and third embodiments, the at least about 700 mg/day ALZ-801 administered to the subject refers to an amount of at least about 750 mg/day ALZ-801, about 750 mg/day ALZ-801, about 700 mg/day to about 1.0 g/day, about 795 mg/day ALZ-801, or about 265 mg ALZ-801 TID.

As part of a fourth embodiment, provided herein is a method of treating Alzheimer's disease in a subject comprising the steps of: a) administering about 400 mg/day to about 600 mg/day (e.g., about 530 mg/day) of ALZ-801 to the subject for a first period of about 50 weeks or more; b) administering at least about 700 mg/day (e.g., about 795 mg/day) of ALZ-801 for a second period of 35 weeks or more; and c) administering at least about 1.0 g/day (e.g., about 1.06 g/day) of ALZ-801 thereafter.

In some aspects of the fourth embodiment, the first period is about 52 weeks or more, about 55 weeks or more, about 60 weeks or more, about 65 weeks or more, about 70 weeks or more, about 75 weeks or more, about 80 weeks or more, 50 weeks to about 80 weeks, 50 weeks to about 75 weeks, 50 weeks to about 70 weeks, 50 weeks to about 65 weeks, about 52 weeks to about 80 weeks, about 52 weeks to about 75 weeks, about 52 weeks to about 70 weeks, or about 52 weeks to about 65 weeks.

In some aspects of the fourth embodiment, the second period is about 40 weeks or more, about 50 weeks or more, about 52 weeks or more, about 55 weeks or more, about 60 weeks or more, about 65 weeks or more, about 70 weeks or more, about 75 weeks or more, about 80 weeks or more, 35 weeks to about 40 weeks, 35 weeks to about 50 weeks, 35 week to about 52 weeks, 35 weeks to about 55 weeks, 35 weeks to about 60 weeks, about 40 weeks to about 50 weeks, about 40 weeks to about 52 weeks, about 40 weeks to about 55 weeks, about 40 weeks to about 50 weeks, about 45 weeks to about 50 weeks, about 45 weeks to about 52 weeks, about 45 weeks to about 55 weeks, about 45 weeks to about 60 weeks, or about 50 weeks to about 55 weeks, or about 50 weeks to about 60 weeks.

As part of a fifth embodiment, provided herein is a method of treating Alzheimer's disease in a subject comprising the steps of: a) administering a first dosage of about 400 mg/day to about 600 mg/day of ALZ-801 to the subject for a first period of time until the subject demonstrates a decreased therapeutic response to the first dosage; and b) administering a second dosage that is at least 1.5 times higher than the first dosage for a second period of time. In some aspects of the fifth embodiment the second dosage is 1.5 to less than 2 times higher than the first dosage.

As part of a sixth embodiment, provided herein is a method of treating Alzheimer's disease in a subject comprising the steps of: a) administering a first dosage of about 400 mg/day to about 600 mg/day of ALZ-801 to the subject for a first period of time until the subject demonstrates a decreased therapeutic response to the first dosage; b) administering a second dosage that is 1.5 to less than 2 times higher than the first dosage for a second period of time until the subject demonstrates a decreased therapeutic response to the second dosage; and c) administering a third dosage that is at least 2 times higher than the first dosage for a third period of time. In some aspects of the sixth embodiment the third dosage is 2 to less than 2.5 times higher than the first dosage.

As part of a seventh embodiment, provided herein is a method of treating Alzheimer's disease in a subject comprising the steps of: a) administering a first dosage of about 400 mg/day to about 600 mg/day of ALZ-801 to the subject for a first period of time until the subject demonstrates a decreased therapeutic response to the first dosage; b) administering a second dosage that is 1.5 to less than 2 times higher than the first dosage for a second period of time until the subject demonstrates a decreased therapeutic response to the second dosage; c) administering a third dosage that is 2 to less than 2.5 times higher than the first dosage for a third period of time until the subject demonstrates a decreased therapeutic response to the third dosage; and d) administering a fourth dosage that is 2.5 times or higher than the first dosage. In some aspects of the seventh embodiment the fourth dosage is 2.5 to less than 3 times higher than the first dosage.

As used herein, “decreased therapeutic response” refers to an observed or measured decrease in a subject's previously observed or measured therapeutic response to ALZ-801 over a period of time. For example, if during treatment with 400 mg/day to about 600 mg/day (e.g., about 530 mg/day) ALZ-801, a subject demonstrates a reversal or a halting of cognitive decline (as compared to prior to such treatment), a decreased therapeutic response would be an observed or measured increase of cognitive decline from those reversed or halted levels. If during treatment with 400 mg/day to about 600 mg/day (e.g., about 530 mg/day) ALZ-801, a subject demonstrates a slowing of cognitive decline (as compared to prior to such treatment), an increase in the rate of cognitive decline would indicate a decreased therapeutic response.

As part of an eighth embodiment, the decreased therapeutic response mentioned herein (including any one of the fourth through sixth embodiments) is demonstrated over a period of at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 12 months, or at least about 1 year. Alternatively, as part of a fourth embodiment, the decreased therapeutic response mentioned herein (including the fourth embodiment) is demonstrated over a period of about 1 week to about 1 year, about 1 month to about 1 year, about 3 months to about 1 year, about 6 months to about 1 year, about 1 week to about 6 weeks, about 1 week to about 5 weeks, about 1 week to about 4 weeks, about 1 week to about 3 weeks, about 2 weeks to about 6 weeks, about 2 weeks to about 4 weeks, or about 2 weeks to about 3 weeks.

As part of a ninth embodiment, a decreased therapeutic response mentioned herein (including any one of the third through eighth embodiments) is demonstrated by a observable decline in one or more activities of daily living, such as reduced motor skills, reduced verbalizations, reduced conversation, reduced comprehension, reduced ability to feed oneself, reduced food consumption, reduced mobility, reduced cooperation with caretakers, increased depression, increased agitation, increased aberrant motor behaviors or increased repetitive movement. Typically, the observable decline in one or more activities of daily living will be made by one or more persons whose interactions with the subject are frequent enough and whose knowledge of the subject's daily activity are sufficiently detailed such that they can make a reasonable assessment of the decline. Such person is typically a caregiver for or a family member of the subject (e.g., a parent, spouse, child, nurse, or other professional caregiver). Such person may also be a physician who is familiar with the subject's daily activity (e.g., as reported by a caregiver or family member).

As part of a tenth embodiment, a decreased therapeutic response mentioned herein (including any one of the third through eighth embodiments) is demonstrated by one or more cognitive tests (e.g., the mini-mental state examination (MMSE), the Mini-Cog test, or the Clinical Dementia Rating scale-sum of boxes (CDR-SB), any improvements or modifications to any of the foregoing, any newly developed cognitive test, or a combination of any of the foregoing).

In one aspect of the tenth embodiment, a decreased therapeutic response mentioned herein is demonstrated by an MMSE decrease of at least two, at least three, at least four, or at least five points over a period of at least one month, at least two months, at least three months, at least four months, at least five months, at least six months, or at least one year. Alternatively, in another aspect of the tenth embodiment, the subject's rate of cognitive decline is demonstrated by an MMSE decrease of at least two, at least three, at least four, or at least five points over a period of about three months to about one year or about six months to about one year. In another alternative aspect of the tenth embodiment, the subject's rate of cognitive decline is demonstrated by an MMSE decrease ranging from two to five points, from two to four points, from two to three, from three to five points, from three to four points, or from four to five points over a period of at least one month, at least two months, at least three months, at least four months, at least five months, at least six months, or at least one year. In yet another alternative aspect of the tenth embodiment, the subject's rate of cognitive decline is demonstrated by an MMSE decrease ranging from two to five points, from two to four points, from two to three, from three to five points, from three to four points, or from four to five points over a period of about three months to about one year or about six months to about one year.

In still another aspect of the tenth embodiment, a decreased therapeutic response mentioned herein is demonstrated by CDR-SB increase of 0.5 to 1 over a period of at least one month, at least two months, at least three months, at least four months, at least five months, at least six months, or at least one year. In still another aspect of the tenth embodiment, the subject's rate of cognitive decline is demonstrated by CDR-SB increase of 0.5 to 1 over a period of about three months to about one year or about six months to about one year.

In certain aspects of the ninth and tenth embodiments, the observations of decline in one or more activities of daily living may be combined with cognitive tests to determine and confirm that the subject is experiencing a decreased therapeutic response. It should be understood that in certain circumstances, conflicting results may be obtained in comparing the results of one or more cognitive tests with observations of one or more activities of daily living. In other words, while a cognitive test may not show an increasing decline, observations of daily living may indicate that such a decline is occurring. Similarly, a cognitive test may suggest an increasing decline, while observations of daily living do indicate any sort of decline. In such situations, the decision to increase the dosage of ALZ-801 may be made by subject's treating physician, optionally in conjunction with the subject's caregivers and family.

As part of an eleventh embodiment, the second dosage amount of ALZ-801 described in fifth, sixth and seventh embodiments is at least about 700 mg/day, at least about 750 mg/day, at least about 795 mg/day, about 700 mg/day to about 1000 mg/day, about 750 mg/day to about 1000 mg/day, about 750 mg/day to about 800 mg/day, or about 795 mg/day. In some aspects of the eleventh embodiment, the second dosage amount of ALZ-801 described in fifth, sixth and seventh embodiments is administered TID. In some aspects of the eleventh embodiment, the second dosage amount of ALZ-801 described in the fifth, sixth and seventh embodiments is about 265 mg ALZ-801 administered TID.

In some aspects of the eleventh embodiment, the third dosage amount of ALZ-801 described in the sixth and seventh embodiments is at least about 1000 mg/day, at least about 1050 mg/day, about 1000 mg/day to less than about 1300 mg/day, about 1000 mg/day to about 1100 mg/day, about 1050 mg/day to less than about 1300 mg/day, about 1050 mg/day to about 1100 mg/day, or about 1060 mg/day. In some aspects of the eleventh embodiment, the third dosage amount of ALZ-801 described in the sixth and seventh embodiments is administered TID. In other aspects of the eleventh embodiment, the third dosage amount of ALZ-801 described in the sixth and seventh embodiments is administered QID. In other aspects of the eleventh embodiment, the third dosage amount of ALZ-801 described in the sixth and seventh embodiments is 265 mg ALZ-801 administered QID.

In some aspects of the eleventh embodiment, the fourth dosage amount of ALZ-801 described in the seventh embodiment is at least about 1300 mg/day, at least about 1325 mg/day, at least about 1400 mg/day, at least about 1450 mg/day, at least about 1500 mg/day, at least about 1550 mg/day, at least about 1590 mg/day, about 1300 mg/day to about 1600 mg/day, about 1325 mg/day to about 1600 mg/day, about 1325 mg/day to about 1590 mg/day, about 1325 mg/day, or about 1590 mg/day. In some aspects of the eleventh embodiment, the fourth dosage amount of ALZ-801 described in the seventh embodiment is administered TID. In other aspects of the eleventh embodiment, the fourth dosage amount of ALZ-801 described in the seventh embodiments is administered QID. In still other aspects of the eleventh embodiment, the fourth dosage amount of ALZ-801 described in the seventh embodiments is administered five or six times per day. In still other aspects of the eleventh embodiment, the fourth dosage amount of ALZ-801 described in the seventh embodiment is 265 mg ALZ-801 administered five times per day. In still other aspects of the eleventh embodiment, the fourth dosage amount of ALZ-801 described in the seventh embodiment is 265 mg ALZ-801 administered six times per day.

As part of a twelfth embodiment, subjects to be treated by the present methods (including any one of the first to eleventh embodiments) have mild to moderate AD. Alternatively, as part of a twelfth embodiment, subjects treated by the present methods (including any one of the first to eleventh embodiments) have moderate to severe AD. In another alternative, as part of a twelfth embodiment, subjects treated by the present methods (including any one of the first to eleventh embodiments) have an MMSE score of 21 or less, 20 or less, 19 or less, 18 or less, 17 or less, 16 or less, or 15 or less.

In some aspects of the twelfth embodiment, subjects treated by the present methods (including any one of the first to eleventh embodiments), have a MMSE score ranging from 15 to 21, 16 to 21, 17 to 21, 18 to 21, 19 to 21, 15 to 20, 16 to 20, 17 to 20, 18 to 20, 15 to 19, 16 to 19, 17 to 19, 15 to 18, or 16 to 18. In yet other aspects of the twelfth embodiment, subjects treated by the present methods (including any one of the first to eleventh embodiments), have an MMSE score of 15, 16, 17, 18, 19, 20, or 21.

As part of a thirteenth embodiment, subjects to be treated by the present methods (including any one of the first to twelfth embodiments) are those who have at least one APOE4 allele, i.e., APOE4 positive or APOE4⁺ subjects. In some aspects of the twelfth embodiment, subjects to be treated by the present methods (including any one of the first to eleventh embodiments) are APOE4 homozygous, i.e. APOE4/4 subjects.

As part of a fourteenth embodiment, ALZ-801 as defined herein (including any one of the first to thirteenth embodiments) is administered to the subject orally.

As part of a fifteenth embodiment, ALZ-801 as defined herein (including any one of the first to fourteenth embodiments) is formulated into a tablet, a capsule, a liquid, an orally dissolving tablet, a sachet, or sprinkles. In some aspects of a fifteenth embodiment, ALZ-801 is formulated into a capsule. In some aspects of a fifteenth embodiment, ALZ-801 is formulated into an instant release capsule. In other aspects of a fifteenth embodiment, ALZ-801 is formulated into an extended release capsule. In still other aspects of a fifteenth embodiment, each capsule of comprises about 265 mg of ALZ-801.

As part of a sixteenth embodiment, if a subject being administered an increased dose of ALZ-801 of at least about 700 mg/day (including any one of the first through fifteenth embodiments) demonstrates unwanted side effects, such as gastric distress or nausea, the dose can be reduced for a period of time to a dose that does not result in such side effects, e.g., an amount higher than the prior dosage level that was being administered to the subject and less than the dosage that caused unwanted side effects, e.g., to greater than about 530 mg/day and less than about 795 mg/day. Once the subject can tolerate the reduced dose of ALZ-801, that dose can be increased back to at least about 700 mg/day. In some aspects of this twenty-third embodiment, the dose increase back to at least about 700 mg/day may be done in a step-wise fashion over several weeks, e.g. increasing the daily dose each week, two weeks, or three weeks, by about 50, about 75, about 100, about 150, about 200, or about 250 mg until the desired dose is reached. In this way, the subject better increases tolerability to the increased dosages.

As part of a seventeenth embodiment (including any one of the first and twelfth through fifteenth embodiments, a ALZ-801 treatment naïve subject who will be receiving a dose of ALZ-801 of at least about 700 mg/day (e.g., at least about 795 mg/day), or who will be ramped up to that dose. In other words, a subject who is not currently receiving any ALZ-801 is initially administered a lower daily dose of ALZ-801, e.g., about 200-300 mg/day for a period of time to determine if the subject experiences any unwanted side effects. Once it is established that the lower dose is tolerated, the daily dose is stepped up (e.g., by about 50, about 75, about 100, about 125, about 150, about 175, about 200, about 250, or about 265 mg) for a period of time (e.g., one week, two weeks, or three weeks) until that stepped up dose is tolerated and the stepping up process is repeated until the desired daily dose is reached.

As part of an eighteenth embodiment is provided herein a pharmaceutical kit for used in any of the aforementioned methods. The kit comprises individual dosages of ALZ-801 to be taken by the subject at one time in accordance with any of the above-mentioned embodiments, separated from one another, e.g., in individual compartments, such as foil-packed or blister-packed unit doses. For example, if the dosage to be taken is 265 mg of ALZ-801 three times per day, each 265 mg dose is present in a separate compartment. The separate compartments may be labeled to indicate the time of day to take the dose (e.g., morning, afternoon and night when the subject is to take ALZ-801 three times per day) and/or the day of the week to take the dose. The kit may further include instructions for taking the doses. The kit may contain, in toto, sufficient dosages for a week, two weeks, three weeks, four weeks, a month, two months, three months, six months, nine months, a year, or more.

In some aspects of the eighteenth embodiment, the dosages of ALZ-801 in the kit are present in multiples of three. Such kits are useful for subject for whom administration of ALZ-801 is TID. In some aspects of the eighteenth embodiment, the dosages of ALZ-801 in the kit are present in multiples of four. Such kits are useful for subject for whom administration of ALZ-801 is QID.

### EXEMPLIFICATION

### Compassionate Use Study

A 72 year old female subject who received a diagnosis of major neurocognitive disorder of the Alzheimer type was treated with ALZ-801 as part of a compassionate use study. The subject was a APOE4 homozygous and had received standard cognitive enhancing medications and was enrolled in a trial of an amyloid immunotherapy agent, but without benefit, and had no treatment options.

The subject was orally administered ALZ-801 at a dose of 265 mg BID for approximately 14 months (61 weeks, 2 days). Up to about the later end of the first 13 months of treatment (about 58 weeks), there was a significant improvement and stabilization of her symptoms. For example, her motor skills improved as she made efforts to brush her teeth, get dressed, and bathe herself. She also was able to hold a phone to her car and was walking and eating better. She was not able or willing to do the above prior to treatment with 265 mg BID ALZ-801. Further, her mood was noticeably more cheerful and content, and her verbal communication advanced. However, by the later end of the first 12-months of treatment, the improvement in the subject's symptoms plateaued and there was a decline in her cognitive status. She became apathetic, less verbal, and could no longer follow instructions. She also exhibited diminished physical activity and worsening in her gait.

At 62 weeks, 6 days of treatment, the dosage of ALZ-801 was increased from 265 mg BID to 265 mg TID. Remarkably, her plateau and decline in cognition ended upon commencement of the 265 mg TID dosage regimen. She was more talkative, happier, and not only completed the tasks she accomplished while on 265 mg BID ALZ-801, but improved beyond that. She understands the context of conversation and behave in a more “normal” or “natural” manner. Overall, there have been clear improvements in her motivation, verbal output, activities of daily living (i.e., cooperative interactions with caretakers, ability to perform minor tasks, etc.) physical activity, mood, and gait. The subject has been taking 265 mg TID ALZ-801 for at least 9 months without out noticeable side effects. A similar plateau or decline in cognitive status as observed with an ALZ-801 dosage of 265 mg BID began to manifest approximately 12 months after the beginning of the 265 mg TID ALZ-801 dosing. The subject remained on 265 mg TID ALZ-801 for approximately three more months (15 months total) until her dosage was increased to 265 mg QID (four time a day) ALZ-801, which she has maintained for the past seven months. Her caretakers report that the new (QID) dosing regime has been well-tolerated with no associated nausea or noticeable side effects. More importantly, the subject's cognitive status and other quality of life measures appear to have improved and continue stable over the seven months period at QID. Her caretakers have noted that the patient's disposition and alertness has improved, she is more responsive, more cooperative and has improved interaction with the caretakers.

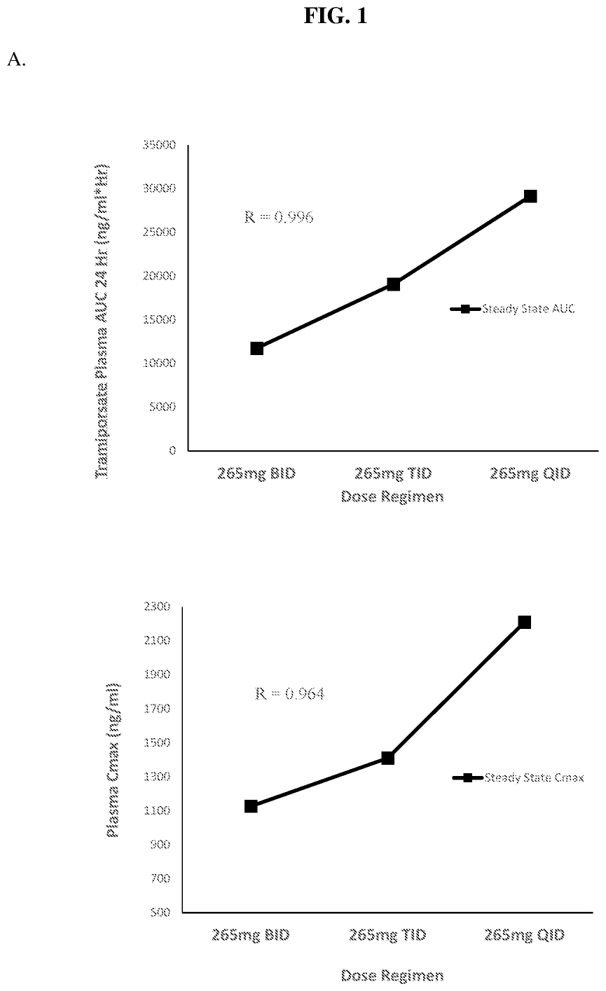

The steady state pharmacokinetics of the TID and QID dosing regimen in the patient has been assayed for area under the curve (AUC) and maximum concentration (Cₘₐₓ) of plasma tramiprosate (the active form of ALZ-801) and compared to BID dosing values in a population of patients in a Phase 2 human clinical trial of ALZ-801. Once the patient reaches steady state on a particular dosing regimen (any time after day 7 of a particular dosing regimen), a blood sample was collected just prior to the administration of a dose of AZL-801 and then again at 1, 2 and 4 hours post-administration of that dose. Plasma was isolated from the sample by centrifugation and the plasma samples were frozen and shipped to a bioanalytical lab for determination of tramiprosate concentrations (active moiety of ALZ-801) and calculation of AUC and Cₘₐₓ. The values obtained from the plasma were then used to calculate projected brain AUC. The results are set forth in Table 1, below and in FIG. 1. The results show dose proportional increases in plasma AUC and Cₘₐₓ based on frequency of dosing.

TABLE 1
Plasma tramiprosate AUC and Cmax for
various daily dosing frequencies.

| | | Expanded | Expanded |
|---|---|---|---|
| Steady-state plasma and | Phase 2 study | access | access |
| projected brain tramiprosate | BID | subject, TID | subject, QID |
| exposure | (n = 23) | (n = 1) | (n = 1) |
| Dose schedule | 265 mg BID | 265 mg TID | 265 mg QID |
| Daily dose (mg) | 530 | 795 | 1060 |
| Plasma Cmax (ng/ml) | 1126 | 1410 | 2210 |
| Plasma AUC24 h (ng/ml*h) | 11747 | 19079 | 29154 |
| Plasma AUC24 h (μM*h) | 84 | 137 | 209 |
| Projected brain AUC24 h | 34 | 55 | 83 |
| (μM*h) | | | |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) that may be cited throughout this application are hereby expressly incorporated herein in their entireties by reference. Unless otherwise defined, all technical and scientific terms used herein are accorded the meaning commonly known to one with ordinary skill in the art.

1. A method of treating Alzheimer's disease in a subject having a MMSE score of 21 or less comprising the step of administering to the subject at least about 700 mg/day ALZ-801.

2. The method of claim 1, wherein the subject is administered about 700 mg/day to about 1.0 g/day.

3. The method of claim 1 or 2, wherein the subject is administered about 795 mg/day ALZ-801.

4. The method of any one of claims 1 to 3, wherein the subject is administered about 265 mg ALZ-801 TID.

5. A method of treating Alzheimer's disease in a subject comprising the steps of:
a. administering about 400 mg/day to about 600 mg/day of ALZ-801 to the subject for a first period of 50 weeks or more; and
b. administering at least about 700 mg/day ALZ-801 thereafter.

6. The method of claim 5, wherein the first period is between about 52 to about 65 weeks.

7. The method of claim 5 or 6, wherein the subject is administered about 530 mg/day of ALZ-801 for the first period.

8. The method of claim 7, wherein the subject is administered about 265 mg of ALZ-801 BID for the first period.

9. The method of any one of claims 5 to 8, wherein the subject is administered about 795 mg/day ALZ-801 thereafter.

10. The method of claim 9, wherein the subject is administered about 265 mg ALZ-801 TID thereafter.

11. The method of any one of claims 1 to 10, wherein the subject has an MMSE score of 21 or less.

12. The method of claim 11, wherein the subject has an MMSE score of 16 or less.

13. The method of claim 11, wherein the subject has an MMSE score of 16 to 21.

14. The method of any one of claims 5 to 10, wherein the subject has an MMSE score of 16 or less prior to administering the at least about 700 mg/day ALZ-801 thereafter.

15. A method of treating Alzheimer's disease in a subject comprising the steps of:
a. administering a first dosage of about 400 mg/day to about 600 mg/day of ALZ-801 to the subject for a first period of time until the subject demonstrates a decreased therapeutic response to the first dosage; and
b. administering a second dosage that is at least 1.5 times higher than the first dosage for a second period of time.

16. The method of claim 15, wherein the second dosage is 1.5 to less than 2 times higher than the first dosage.

17. The method of claim 16, wherein the second period of time is until the subject demonstrates a decreased therapeutic response to the second dosage; and wherein the method further comprises administering a third dosage that is at least 2 times higher than the first dosage for a third period of time.

18. The method of claim 17, wherein the third dosage is 2 to less than 2.5 times higher than the first dosage.

19. The method of claim 18, wherein the third period of time is until the subject demonstrates a decreased therapeutic response to the third dosage; and wherein the method further comprises administering a fourth dosage that is 2.5 times or higher than the first dosage.

20. The method of claim 19, wherein the fourth dosage is 2.5 to less than 3 times higher than the first dosage.

21. The method of any one of claims 15-20, wherein the second dosage amount of ALZ-801 is at least about 700 mg/day.

22. The method of claim 21, wherein the second dosage amount of ALZ-801 is about 795 mg/day.

23. The method of claim 21 or 22, wherein the second dosage amount of ALZ-801 is administered TID.

24. The method of any one of claims 17-23, wherein the third dosage amount of ALZ-801 is at least about 1000 mg/day.

25. The method of claim 24, wherein the third dosage amount of ALZ-801 is about 1060 mg/day.

26. The method of claim 24 or 25, wherein the third dosage amount of ALZ-801 is administered TID or QID.

27. The method of claim 26, wherein the third dosage amount of ALZ-801 is administered QID.

28. The method of any one of claims 19-27, wherein the fourth dosage amount is at least about 1300 mg/day.

29. The method of claim 28, wherein the fourth dosage amount is about 1325 mg/day to about 1590 mg/day.

30. The method of claim 29, wherein the fourth dosage amount of ALZ-801 described in the seventh embodiment is administered TID, QID, five times per day, or six times per day.

31. The method of any one of claims 15 to 30, wherein the decreased therapeutic response is demonstrated over a period of at least about two weeks, about two to about three weeks, at least about 6 months, or about 6 months to about 1 year.

32. The method of any one of claims 15 to 31, wherein the decreased therapeutic response is demonstrated by one or more of reduced motor skills, reduced verbalizations, reduced conversation, reduced comprehension, reduced ability to feed oneself, reduced food consumption, reduced mobility, reduced cooperation with caretakers, increased depression, increased agitation, increased aberrant motor behaviors or increased repetitive movement.

33. The method of any one of claims 15 to 31, wherein the decreased therapeutic response is demonstrated by one or more cognitive tests.

34. The method of claim 33, wherein the one or more cognitive tests are selected from the mini-mental state examination (MMSE), the Mini-Cog test, and the Clinical Dementia Rating scale-sum of boxes (CDR-SB), or a combination thereof.

35. The method of claim 34, wherein the decreased therapeutic response is demonstrated by one or more of:
a. an MMSE score decrease of 4 to 5 over a period of about 6 months to about one year; or
b. a CDR-SB increase of 0.5 to 1 over a period of about 6 months to about one year.

36. The method of any one of claims 1 to 35, wherein the subject is APOE4⁺.

37. The method of claim 36, wherein the subject is APOE4 homozygous.

38. The method of any one of claims 1 to 37, wherein the ALZ-801 is formulated into a tablet, a capsule, a liquid, an orally dissolving tablet, a sachet, or sprinkles.

39. The method of claim 38, wherein the ALZ-801 is formulated into a capsule.

40. A pharmaceutical kit comprising individual dosages of ALZ-801 separated from one another, wherein the dosages of ALZ-801 in the kit are present in multiples of three or multiples of four.
